(19) United States
(12) Patent Application Publication
(10) Pub. No.: US 2026/0174521 A1
(43) Pub. Date: Jun. 25, 2026

(54) CRANIAL ACCESS ASSEMBLY AND METHOD OF USING THE SAME

(71) Applicant: Monteris Medical Corporation, Minnetonka, MN (US)

(72) Inventors: Mark Andrew Grant, Winnipeg (CA); Walter John Dobrovolny, Saint Paul, MN (US)

(21) Appl. No.: 19/126,429

(22) PCT Filed: Nov. 2, 2023
(86) PCT No.: PCT/US2023/078536

§ 371 Date: May 1, 2025

Related U.S. Application Data
(60) Provisional application No. 63/382,092, filed on Nov. 2, 2022.

(51) Int. Cl. A61B 90/11; A61B 17/02; A61B 90/14

(52) U.S. Cl. CPC A61B 90/11; A61B 17/0218; A61B 90/14

(57) ABSTRACT

A cranial access assembly is disclosed. The cranial access assembly includes a cranial bolt and a trajectory adjustment guide. The cranial bolt includes a channel configured for receiving a neurosurgical tool. The trajectory adjustment guide includes a distal portion and a proximal portion. The distal portion is configured to be received within the channel at one of a plurality of angular positions. The proximal portion has a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel.

### CROSS REFERENCE TO RELATED APPLICATIONS

This application claims priority to U.S. Application No. 63/382,092, filed Nov. 2, 2022, which is incorporated herein by reference in its entirety.

### FIELD OF THE DISCLOSURE

The present disclosure generally relates devices and methods for gaining access to the interior of human and animal brains. Specifically, the disclosure relates to cranial bolts or screws for securing medical devices to the skull and for providing access to an interior of the skull, and methods of operation thereof.

### BACKGROUND OF THE DISCLOSURE

Cranial access devices such as cranial bolts are used to secure medical devices to the head and control the depth and/or trajectory of such medical devices inserted into the brain within the skull. The medical devices can include, for example, catheters, neurosurgical tools, probe drivers, or probes. The medical device is introduced into a lumen or hole of the access device and into a corresponding hole in the skull.

The medical device is secured to the skull by threading the cranial access assembly into the cranium and then attaching or coupling the medical device to the cranial access device. For example, an interventional procedure in the subject's brain may involve drilling a burr hole into a subject's skull, mounting a cranial access assembly on the subject's skull, and guiding an instrument (e.g., a catheter, a needle, a cannula, an electrode, or other device) to the desired target within the subject via the cranial access device, such as by using pre-operative or live images from an imaging modality (e.g., magnetic resonance (MR), computerized tomography (CT), position emission tomography (PET), ultrasound, etc.) in an image-guided procedure. Accurate guidance is desirable, particularly for an interventional procedure in the brain, where millimeter or sub-millimeter accuracy of the instrument location may be desirable.

A typical workflow includes generally, identifying a tissue in a subject to be treated, planning one or more trajectories for treating the tissue, preparing the subject and components for the treatment, and performing the treatment. In pre-planning of a treatment of a subject, pre-treatment image data (e.g., MR, CT, PET, ultrasound, etc.) is loaded and co-registered. An intended treatment region of interest(s) (ROI)(s) and initial trajectory(s) are created and established as desired. Based on the ROI and the initial trajectory, a probe entry location into the skull is identified, and a burr hole or a twist drill hole may be created for both brain access and affixation of the cranial access device. Specifically, the cranial access assembly(e.g., a twist bolt) may be affixed (e.g., by threading) at the identified entry point generally along the identified trajectory to create a guideway to insert a medical device and constrain its insertion to the identified trajectory as it is inserted through the access device.

However, the trajectory planned in the operating room (using pre-treatment image data) may not be sufficiently accurate when the subject is moved to an imaging modality (e.g., MR) and the actual medical device location is identified using real-time imaging. Specifically, the actual location of the medical device may not coincide with the previously planned trajectory for various reasons. Furthermore, even if the trajectory is accurate and no adjustment is needed, the planned treatment may require adjustments (e.g., when the final ablation volume is not conformal to a ROI during laser ablation) leading to changes in the trajectory of the medical device. Since cranial access devices are typically rigid whose trajectory cannot be altered after being affixed to skull, a new cranial hole and a cranial access assembly must be inserted for aligning the medical device with the previously planned and/or new trajectory, which in turn requires movement of the subject from the imaging modality to the operating room for such a procedure.

The current disclosure describes devices and methods directed towards solving some of the issues discussed above.

### SUMMARY OF THE DISCLOSURE

Disclosed scenarios include cranial access assembly and methods of using the same. A cranial access assembly is disclosed. The cranial access assembly may include a cranial bolt and a trajectory adjustment guide. The cranial bolt may include a channel configured for receiving a neurosurgical tool. The trajectory adjustment guide may include a distal portion and a proximal portion. The distal portion may be configured to be received within the channel at one of a plurality of angular positions. The proximal portion may have a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel.

In one or more embodiments, the cranial bolt may include a cranial bolt distal portion configured for attachment to a skull of a subject. Optionally, the cranial bolt distal portion may include threads.

In one or more embodiments, an inner surface of the channel may include one or more locking features that lock the distal portion at that one of the plurality of angular positions when the distal portion is inserted within the channel. Optionally, the inner surface of the channel may include a hex shape cross section that provide six angular positions of the distal portion within the channel.

In certain other embodiments, an outer surface of the distal portion may include one or more locking features that lock the distal portion at that one of the plurality of angular positions when inserted within the channel.

In yet other embodiments, the trajectory adjustment guide may include a locking element configured to lock the distal portion at that one of the plurality of angular positions when the distal portion is inserted within the channel. Optionally, the locking element can include a screw.

Optionally, a length of the cranial bolt is about 10 mm to about 45 mm. Additionally and/or alternatively, an angular offset between the trajectory adjustment channel and the channel when the trajectory adjustment guide is at least partially inserted within the channel is about 0.5° to about 30°.

In various embodiments, a diameter of the trajectory adjustment channel may be configured to hold the neurosurgical tool via a friction fit.

In some embodiments, at least one of the cranial bolt or the trajectory adjustment guide may include one or more interfacing features configured to interface with a depth stop that is configured to set a depth of the neurological tool within a cranial cavity of a subject.

In various scenarios, a trajectory adjustment guide for adjusting a trajectory of a neurosurgical tool is disclosed. The trajectory adjustment guide may include a distal portion and a proximal portion. The distal portion may be configured to be received within a channel of a cranial bolt at one of a plurality of angular positions, the cranial bolt being affixed to a skull of a subject along a pre-planned trajectory. The proximal portion may have a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel.

In certain other embodiments, an outer surface of the distal portion may include one or more locking features that lock the distal portion at that one of the plurality of angular positions when inserted within the channel.

In yet other embodiments, the trajectory adjustment guide may include a locking element configured to lock the distal portion at that one of the plurality of angular positions when the distal portion is inserted within the channel. Optionally, the locking element can include a screw.

In various embodiments, an angular offset between the trajectory adjustment channel and the channel when the trajectory adjustment guide is at least partially inserted within the channel is about 0.5° to about 30°. Optionally, a diameter of the trajectory adjustment channel may be configured to hold the neurosurgical tool via a friction fit.

Optionally, the trajectory adjustment guide may include one or more interfacing features configured to interface with a depth stop that is configured to set a depth of the neurological tool within a cranial cavity of a subject. Optionally, the one or more interfacing features may include a slot configured to receive an interface ring of the depth stop. Optionally, the symmetrical locking blocks may be formed from a rigid material to prevent overtightening of the depth stop channel around the neurosurgical tool.

In various scenarios, a floating depth stop for controlling a depth of a neurosurgical tool inserted within a cranial cavity via the cranial access assembly of claim 1 is disclosed. Optionally, the depth stop may include two symmetrical locking blocks that define a depth stop channel. Additionally and/or alternatively, an interfacing ring configured to interface with at least one of the cranial bolt or the trajectory adjustment guide may be included within the floating depth stop. A sleeve surrounding the channel may also be provided within each of the locking blocks.

In some embodiments, the floating depth stop may include a clamp configured for pushing the deformable locking elements around the neurosurgical tool within the depth stop channel to lock an insertion depth of the neurosurgical tool.

In one or more scenarios, a surgical kit including the cranial access assembly discussed above is disclosed. The surgical kit may include a plurality of trajectory adjustment guides, each having a different angular offset between the channel and the corresponding trajectory adjustment channel. Optionally, a floating depth stop configured to set a depth of insertion of the neurosurgical tool may be included in the surgical kit. Additionally and/or alternatively, the surgical kit may include the neurosurgical tool. Other components such as, without limitation, a plurality of reducing tubes configured to reduce a diameter of the trajectory adjustment channel, a plurality of cranial bolts each having a different channel diameter capable of accepting trajectory adjustment guides of varying diameters, or the like may be included in the surgical kit.

In various scenarios, a method for adjusting the trajectory of a neurosurgical tool using a cranial access assembly is disclosed. The methods may include affixing a cranial bolt that includes a channel configured for receiving a neurosurgical tool to a skull of a subject along a first trajectory, inserting the neurosurgical tool within the channel the cranial bolt, determining a trajectory of the neurosurgical tool, and identifying from a plurality of trajectory adjustment guides in response to determining that the trajectory does not align with the first trajectory or will not cause the neurosurgical tool to reach a target site within a cranial cavity. A trajectory adjustment guide may be configured to adjust the trajectory to align with the first trajectory or cause the neurosurgical tool to reach the target site within the cranial cavity. The identified trajectory guide may include a distal portion configured to be received within the channel at one of a plurality of angular positions that is configured to adjust trajectory, and a proximal portion having a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel. The methods may also include inserting the identified trajectory adjustment guide within the channel, and advancing the neurosurgical tool within the trajectory adjustment channel. Optionally, the methods may also include controlling a depth of the neurosurgical tool using a floating depth stop.

### BRIEF DESCRIPTION OF THE DRAWINGS

FIG. 1 is an elevation view of the cranial access assembly of the present invention installed in a subject's skull.

FIG. 2 illustrates an example cranial bolt of the cranial access assembly of FIG. 1.

FIG. 3A illustrates an example trajectory adjustment guide of the cranial access assembly of FIG. 1.

FIG. 3B illustrates a cross-sectional view of an example cranial access assembly.

FIG. 3C illustrates a cross-sectional view of an example cranial access assembly

FIG. 4 illustrates an example fiducial marker.

FIGS. 5A and 5B illustrate cross sectional views of an example depth stop, FIG. 5C illustrates an exploded view of the depth stop.

FIG. 5D illustrates an example depth stop interfaced with a trajectory adjustment guide.

FIG. 5E illustrates an example depth stop interfaced with a cranial bolt.

FIG. 6. illustrates a schematic representation of example of hardware included in various components of the systems of this disclosure.

### BRIEF DESCRIPTION OF DISCLOSED EMBODIMENTS

The devices and methods of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, proximal, distal, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. It should be noted that the terms “proximal” and “distal” as used herein, are intended to refer to a direction toward (proximal) and away from (distal) a surgeon or other user of the device.

While the examples provided in this disclosure generally relate to cranial access assemblies for cranial access, disclosed systems and methods of this disclosure may be used for any other bone through which it is desirable to pass a catheter, probe, or other similar device, such as, but not limited to, the spinal vertebrae, hip, or the like. For example, disclosed embodiments may be used for access to the vertebral body of the spinal vertebra without deviating from the principles disclosed herein.

Cranial bolts are commonly utilized in holes (burr and/or twist drill) in the skull of a subject for subsequent medical device placements (e.g., catheter, electrodes, neurosurgical tools, probe drivers, probes, laser delivery probes, etc.). However, as discussed above, currently available cranial bolts are rigid that do not allow for any changes to the trajectory of the medical device after insertion of the cranial bolt into the hole in the skull. The current disclosure describes a cranial access assembly and methods of use thereof that can be used for alignment with a pre-planned trajectory (in case of alignment errors) and/or modifications to a trajectory that has been pre-planned (the modifications made based on an updated trajectory and through a cranial bolt already inserted into the skull at an entry point in accordance with the pre-planned trajectory). Embodiments may be used with dedicated medical devices or systems that are designed anew, or with preexisting systems. For example, embodiments may be used with medical devices or systems like the ones shown in U.S. Pat. Nos. 9,510,909 and 10,327,830, the disclosures of which are incorporated herein by reference in entirety.

The devices and methods disclosed herein aim to improve upon at least one of the aforementioned problems. However, it shall be understood that the disclosure herein is not limited to merely solving these specific problems. Additionally, while the devices and techniques disclosed herein are described with respect to a human body or subject, it is understood that the devices and techniques may in suitable circumstances be applied to a non-human subject (i.e., in veterinary medicine).

In various implementations, the current disclosure describes a cranial access assembly including a cranial bolt and a trajectory adjustment guide. A medical device may be guided through a contiguous lumen (i.e., a lumen formed by a first channel of the cranial bolt and a second channel of the trajectory adjustment guide) for accessing the cranium and/or dura of the subject. In various embodiments, the internal diameter(s), shape, length, or other physical parameters of the cranial bolt channel may be defined such that a medical device inserted through the contiguous lumen does not make physical contact (or makes minimal physical contact) with the walls of the cranial bolt channel along any of the one or more trajectories defined by the trajectory adjustment guide(s) configured to be used with the cranial bolt.

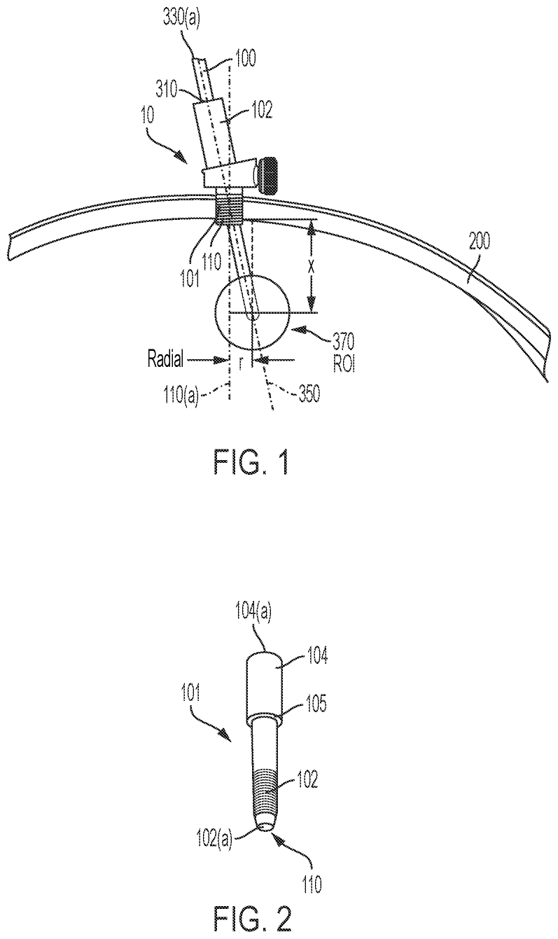

Referring now to the drawings, and in particular to FIGS. 1-4, an example cranial access assembly 10 is shown and described. The cranial access assembly 10 can be used to facilitate a medical device 100 to access the cranial cavity of a subject, and for adjustment or control of trajectory of the medical device. Specifically, the cranial access assembly 10 is a skull cranial access assembly that is designed to provide a stable platform for inserting, attaching, manipulating, and/or otherwise coupling medical devices or instruments within a subject along a desired trajectory. Referring to FIG. 1, a cranial access assembly 10 including a cranial bolt 101 and a trajectory adjustment guide 102 is illustrated secured to a skull 200 of a subject, according to the present disclosure.

The cranial bolt 101 is configured to be inserted into a hole formed in a subject's skull, and for allowing a medical device to access the cranial cavity, via a channel or lumen included in the cranial bolt. Typically, the hole is drilled into a subject's skull using a twist drill and a threaded portion of the cranial bolt is screwed directly into the hole. Examples of such cranial bolts are described in, for example, U.S. patent application Ser. No. 17/661,241 entitled “Cranial Access Device” and filed Apr. 28, 2022 and in U.S. patent application Ser. No. 15/573,103 entitled “Apparatus and Method for Neurological Intervention” and filed Mar. 11, 2016, the contents of which are incorporated herein by reference in its entirety.

As shown in FIG. 2, the cranial bolt 101 may include a distal threaded portion 102 and a proximal non-threaded portion 104. A central passageway or channel 110 extends through the length of the cranial bolt 101 (i.e., between a distal end 102(a) and an opposing proximal end 104(a)) and may have a cross section of any geometric shape, e.g. circular, triangle, square, hexagon, and pentagon, or combinations thereof (for example, the passageway near the proximal end may have a hexagonal cross-section while the passageway near the distal end may have a circular cross-section). The diameter of the channel 110 may be uniform throughout the length of the channel and/or may vary (e.g., may be different in the proximal portion and the distal portion). Additionally and/or alternatively, the distal end 102(a) of the threaded portion 102 may not include any threads to provide the ability of inserting the bolt into the skull of the subject in a seamless manner, when the skull wall is too thin to use a fully threaded distal end. In various embodiments, an internal diameter of the channel at a proximal end of the bolt is sized to accept an instrument for neurological intervention that can access the target tissue through the distal end of the bolt channel (with or without the trajectory adjustment guide attached thereto). The proximal portion of the cranial bolt may be configured to (e.g., may have dimensions configured to) interface with the trajectory adjustment guide and/or the adjustable depth stop (as discussed below). Optionally, the proximal portion and/or proximal end of the bolt is dimensioned in a manner such that multiple bolts can be placed in close proximity of one another. The distal portion of the bolt is designed in a manner such that an internal diameter of the channel at the distal portion of the bolt is configured to allow passage of various instruments, received via the proximal portion, into a cranial cavity. Additionally, the outside diameter of the distal portion of the bolt may be configured to prevent injuries to the skull and minimize the diameter of the drilled hole in the skull of the subject. In various embodiments, the parameters of the cranial bolt channel (internal diameter(s), shape, length, or other physical parameters) may be defined such that a medical device inserted through the channel via a trajectory adjustment guide does not make physical contact (or minimal physical contact) with the walls of the channel along any of the trajectories defined by the trajectory adjustment guide(s) configured to be used with the cranial bolt (in order to prevent interference with the medical device, via the channel walls). For example, a total length of the cranial bolt 101 between the proximal end and the distal end is about 10 mm to about 45 mm, about 15 mm to about 40 mm, about 20 mm to about 35 mm, about 25 mm to about 30 mm, about 15 mm, about 20 mm, about 25 mm, about 30 mm, about 35 mm, about 37 mm to about 43 mm, about 38 mm to about 42 mm, about 15 mm, about 18 mm, about 20 mm, about 25 mm, about 40 mm, about 41 mm, about 42, or the like. The internal diameter of the channel within the proximal portion of the cranial bolt 101 may be about 4-8 mm, 7.5-11 mm, about 8-10.5 mm, about 8-10 mm, or the like. The internal diameter of the channel within the distal portion of the cranial bolt 101 may be about 1-2 mm, 1.6-2.2 mm, 2-4 mm, 3.3-5 mm, 5-8 mm, 8-10 mm, or the like. It must be appreciated that each of the above described exemplary dimensions can be varied as long as the resultant bolt is not inconsistent with the description herein.

A medical device (e.g., a neurosurgical device such as a probe or laser probe) can be inserted into central passageway 110 for access to target tissue. For example, the central passageway 110 of the bolt 101 is sized to accept an instrument or a medical device for neurological intervention at the proximal end and that can access the target tissue through the distal end of the bolt 101.

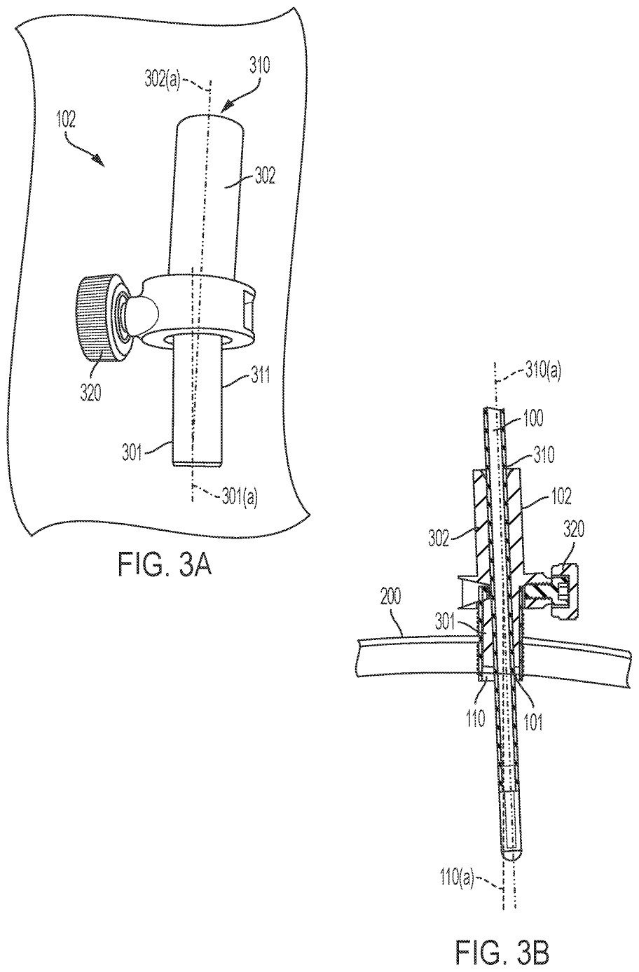

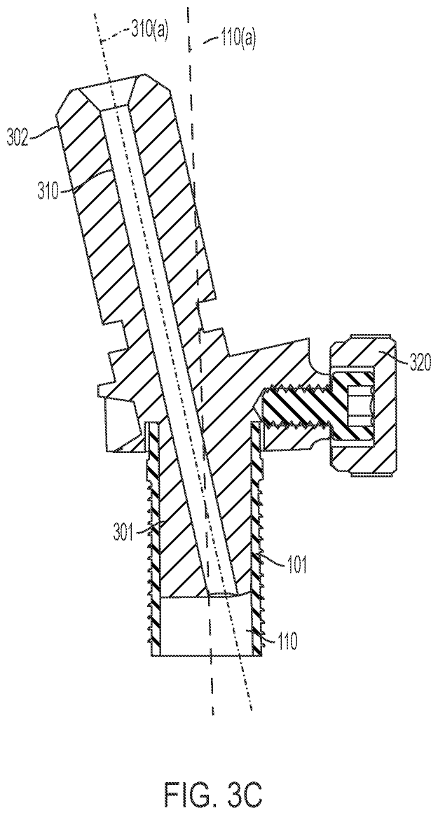

Referring now to FIG. 3A, an example trajectory adjustment guide 102 is illustrated. FIG. 3B illustrates a cross-sectional view of an example trajectory adjustment guide inserted into the cranial bolt. FIG. 3C illustrates a cross-sectional view of another example trajectory adjustment guide inserted into the cranial bolt.

As shown in FIGS. 1 and 3A-3C, the trajectory adjustment guide 102 includes a distal portion 301 configured for insertion (at least partially) within the cranial bolt channel 110, and a proximal portion 302. The proximal portion has a longitudinal axis 302(a) (FIG. 3A) that is angularly offset (or tilted) with respect to the longitudinal axis 301(a) of the distal portion 301. In other words, the distal portion 301 is formed at an angle with respect to the proximal portion 302.

Further, as shown in FIGS. 3B and 3C, a contiguous passageway (i.e., trajectory adjustment channel 310) is formed within and across the entire length of the trajectory adjustment guide 102, where the channel is substantially parallel to (or aligns with) the longitudinal axis 302(a) of the proximal portion 302 while being angularly offset from the longitudinal axis 301(a) of the distal portion. When the distal portion 301 of the trajectory adjustment guide 102 is inserted coaxially into the cranial bolt channel 110, the longitudinal axis 301(a) of the distal portion 301 aligns with (or is parallel) to the axis 110(a) of the cranial bolt channel 110 while the proximal portion 302 is disposed at an angle with respect to the longitudinal axis 110(a) of the cranial bolt channel 110. As such, when the distal portion 301 of the trajectory adjustment guide 102 is inserted coaxially into the cranial bolt channel 110, the trajectory adjustment channel 310 is angularly offset with respect to the cranial bolt channel 110 (as shown in FIG. 3B). The angular offset can be about 0.5-30°, 1-29°, about 2-28°, about 3-27°, about 4-26°, about 5-25°, about 6-24°, about 7-23°, about 8-22°, about 9-21°, about 10-20°, about 11-19°, about 12-18°, about 13-17°, about 14-16°, about 0.5-10°, about 1-9°, about 2-8°, about 3-7°, or the like. The angular offset allows for an adjustment in the trajectory of a medical device inserted into the cranial cavity via the trajectory adjustment channel 310 compared to when the medical device is inserted directly into the cranial bolt channel 110.

In various embodiments, an internal diameter of the trajectory adjustment channel 310 is configured and sized to accept an instrument or a medical device for neurological intervention and/or access to a target tissue. Optionally, a medical device (e.g., a neurosurgical device such as a probe) can be inserted into the trajectory adjustment channel 310 and be held in place by friction, compression fitting, or the like. Optionally, for medical devices that have a diameter that is less than that of the trajectory adjustment channel 310 such that they cannot be held in place by friction or compression fitting (e.g., biopsy needles, SEEG electrodes, catheters, etc.), a reducing tube having a diameter smaller than that of the channel and that can provide friction or compression fit for the medical device may be inserted into the trajectory adjustment channel 310 so as to reduce the diameter of the trajectory adjustment channel 310 for receiving the medical device. The internal diameter of the reducing tube may be configured based on the size/diameter of a medical device.

In various embodiments, at least a portion of the distal portion 301 of the trajectory adjustment guide 102 includes an outer surface profile that is complementary in shape to the inner geometry of the channel 110 of the cranial bolt 101. Moreover the diameter of the outer surface profile is configured such that the distal portion 301 can be inserted at least partially into the channel 110 of the cranial bolt 101 and retained therein by compression fitting or friction. Optionally, the distal portion 301 may be rotated to a plurality of rotational positions within the channel 110 such that the channel 310 may be moved to a plurality of angular positions with respect to the longitudinal axis 110(a) of the channel 110. As such, a trajectory of the medical device inserted into the cranial cavity via the channel 310 may be adjusted to a plurality of angular positions within a plane perpendicular to the longitudinal axis 110(a) of the channel 110. Optionally, the distal portion 301 of the trajectory adjustment guide 102 and/or the channel 110 may include one or more locking features that interface with each other. This allows for to the distal portion 301 to be inserted and/or locked into position within the channel 110 at a desired one of the plurality of angular rotations. For example, the channel 110 may include an internal hex shape cross section with the surface profile of the distal portion 301 having a complementary hex shape (such as that of a nut and socket) allowing for six rotational positions of the distal portion relative to the channel (each being 60° apart). Optionally, the hex shape cross section may be included on a tab provided within the cranial bolt channel that interfaces with complementary hex shape of the distal portion. Other shapes other than a hex shape such as square, triangle, pentagon, etc. are within the scope of this disclosure. In other example, the channel 110 may include tabs that interface with holes included in the surface profile 311 at different rotational positions (or vice versa). Any other now or hereafter known features are within the scope of this disclosure.

Optionally, the trajectory adjustment guide 102 may also include a locking mechanism (e.g., a thumbscrew 320) for clamping and/or locking the distal portion 301 at a desired angular position within the channel 110. The thumbscrew 320 may be suitably tightened to clamp the distal end 301 at a desired angular position within the channel 110.

In various embodiments, a range of trajectory adjustment provided by the trajectory adjustment guide 102 may depend on, for example, the depth of the target (or ROI) within the cranial cavity, the angular offset of the channel 310 relative to cranial bolt channel 110, an angular position of the distal portion 301 of the trajectory adjustment guide 102 within the cranial bolt channel 110, an internal diameter of the cranial bolt channel 110, or the like. For example, as shown in FIG. 1, the channel 310 of the trajectory adjustment guide 102 is angularly offset from the channel 110 of the cranial bolt 101 to allow for an adjustment of the probe trajectory (at multiple angular positions) within a radius “r” (e.g., about 5-8 mm, about 6-7 mm, about 6 mm, about 7 mm, etc.) from the original trajectory 350 (along to the longitudinal axis 110(a) of the cranial bolt 101) at a depth of “x” (e.g., about 10-20 mm, 20-30 mm, about 22-28 mm, about 24-26 mm, about 24 mm, about 25 mm, about 26 mm, about 27 mm, etc.) to the ROI 370 and within a plane perpendicular to the longitudinal axis 110(a).

Optionally, a surgical kit may include the cranial bolt along with a plurality of trajectory adjustment guides, each having a distinct angular offset between the longitudinal axes 301(a) and 302(a) so as to provide a wider range of trajectory adjustments (i.e., a plurality of radii “r”) for a given depth “x” of the target or ROI. Such a kit may provide a range of trajectory adjustment options for achieving a desired trajectory adjustment. For example, the angular offset of the trajectory adjustment guide shown in FIG. 3B is different from that of the trajectory adjustment guide shown in FIG. 3C, and such one or more trajectory adjustment guides may be included in a surgical kit. Optionally, the surgical kit may also include one or more reducing tubes discussed above. Optionally, the surgical kit can include one or more cranial bolts of varying diameter thereby allowing a wider range of trajectory adjustment guides to be used. The distal portion of the various trajectory adjustment guides within the kit would be configured for co-axial insertion into the diameter of the cranial bolt its intended for.

The trajectory adjustment guide of this disclosure may be provided in a plurality of configurations (i.e., different dimensions and/or angular offsets), where each of them can be used within the same cranial bolt. Such configurations of the trajectory adjustment guide provides different dimensions and/or different angular offsets allowing for a wider range of adjustability depending on the offset angle within the same cranial bolt channel. Optionally, the cranial bolt can also have a plurality of configurations for providing a range of diameters that are configured to receive a wide range of adjustable trajectory guides. For example, trajectory adjustment guides with a larger angular offset may require a cranial bolt of larger diameter to allow clearance of any medical device being deployed within the channel of such a trajectory adjustment guide. Such scenarios suggest a range of options of the cranial access assembly selectable by the user depending on the extent of adjustability desired. This may, optionally, include trajectory adjustment guides that have an angular offset of zero (or close to zero) degrees in which case its longitudinal axis is colinear with the axis of the cranial bolt.

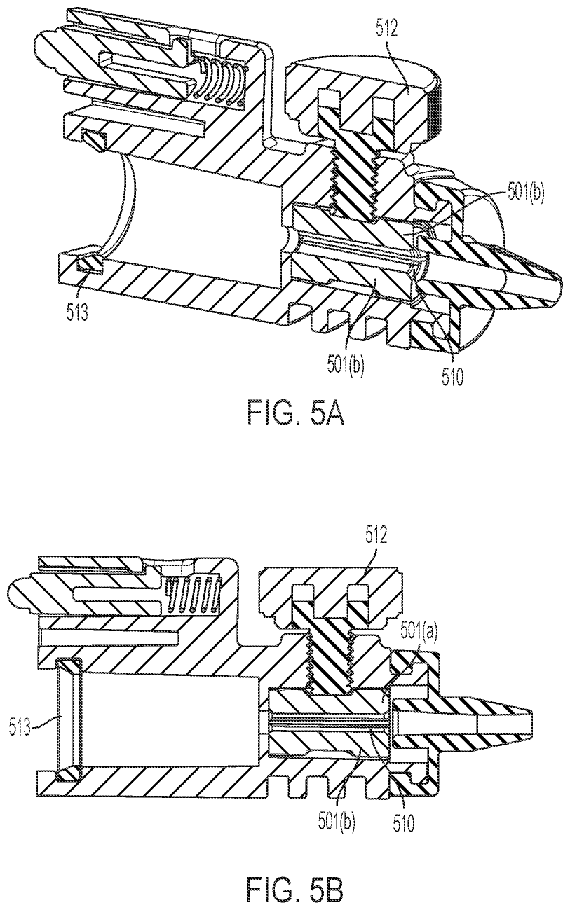

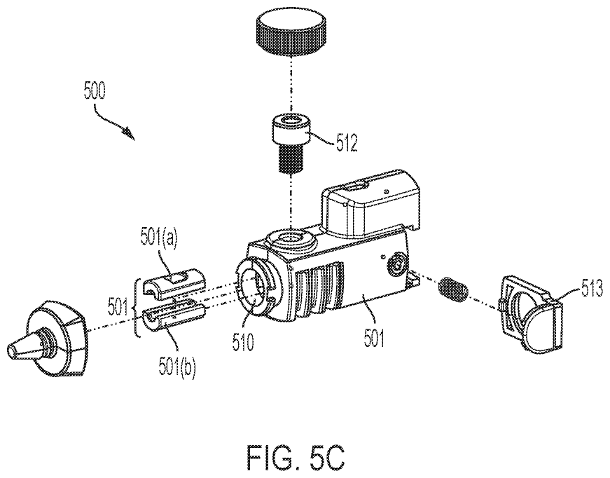

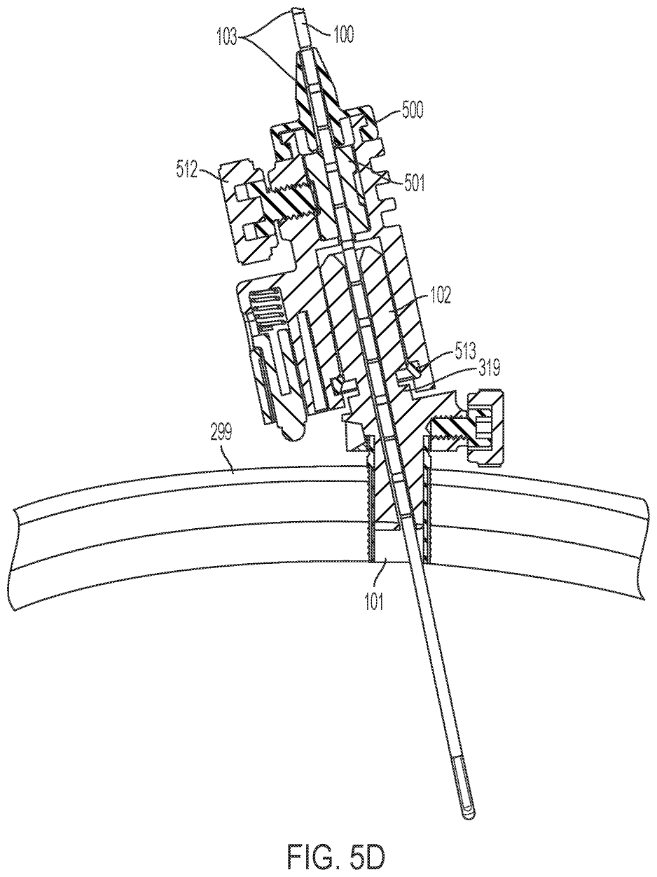

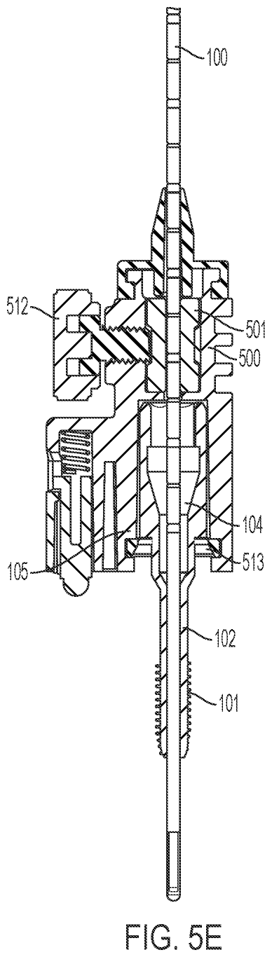

A medical device (e.g., a probe) may be inserted into the channel of the cranial bolt directly and/or via the trajectory adjustment guide for insertion ofthe medical device into the skull. Optionally, a depth stop (discussed below with respect to FIG. 5) may be provided to lock the inserted medical device at a certain depth within the cranial access assembly. An improperly set depth can cause the probe tip to be delivered short of or deeper than intended/planned, which may lead to subject injury, and a depth stop may be used to prevent such errors and/or injury.

In some embodiments, a user may select an appropriate trajectory adjustment guide (from a plurality of trajectory adjustment guides) and/or the corresponding angular position of the distal portion of the identified trajectory adjustment guide that will provide a desired trajectory adjustment.

Additionally and/or alternatively, the system includes a controller including processing circuitry and a memory having instructions stored thereon, where the instructions, when executed by the processing circuitry, cause the processing circuitry to determine, based on imaging (e.g., MR, CT, etc.), a required adjustment to the trajectory of the medical device. For example, the controller may determine that the actual trajectory of a medical device through a cranial bolt is different from a pre-planned trajectory, and determine the required adjustment based on the difference. In another example, the controller may determine that the actual trajectory of a medical device through a cranial bolt may not result in the desired treatment outcome (e.g., an ablation volume of an ROI) and determine a new trajectory for achieving the desired treatment outcome. The controller may also determine the required adjustment between the initial and updated trajectory. The controller may also track the depth stop of the probe and/or determine an accurate depth stop location.

The controller may further, based on the required adjustment, identify a trajectory adjustment guide that will provide the determined adjustment (e.g., from amongst a plurality of trajectory adjustment guides having different offsets and included within a surgical kit). The controller may also determine an angular position of the distal portion of the identified trajectory adjustment guide that will provide the determined adjustment.

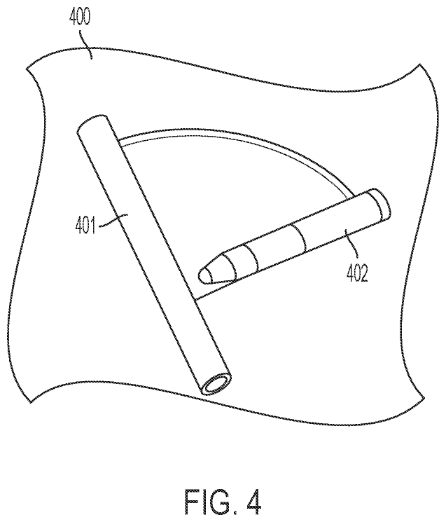

In various embodiments, a radio opaque fiducial marker 400 (as shown in FIG. 4) may be provided (e.g., within a surgical kit including one or more trajectory adjustment guides and cranial bolts). The fiducial marker may be L-shaped including a longitudinal portion 401 and a transverse portion 402 substantially perpendicular to the longitudinal portion. Optionally, the fiducial marker can be another shape where the angle between the longitudinal portion and the transverse portion is known. A distal end of the longitudinal portion 401 may be configured for insertion into the cranial bolt channel in a coaxial manner (i.e., the axis of the longitudinal portion 401 substantially aligns with the axis 110(a) of the channel 110). The fiducial marker 400 is made of a material that is visible within the imaging modality (e.g., CT, MR etc.) such that the axis 110(a) of the channel 110, as well as a transverse axis (i.e., a perpendicular direction) along a known angle relative to the cranial bolt (e.g., 0 degree position) can be identified. Upon identification of the two directions, any now or hereafter known geometrical methods may be used to identify, based on a required adjustment to trajectory, the suitable trajectory adjustment guide and/or an angular position to achieve a desired adjustment to a trajectory at a specific target depth.

The system may output the determined trajectory adjustment, an identification of the trajectory adjustment guide and/or the angular position to achieve the desired trajectory adjustment to a user (e.g., via a display). The user may select the identified trajectory adjustment guide and insert it within an already inserted cranial bolt at the determined angular position, and thus be able to adjust the trajectory of the medical device using an already inserted cranial bolt without having to create another hole or insert another cranial bolt. This also allows for adjustment of trajectory without movement of the subject to an operating room, while being continually imaged in an imaging modality (e.g., in real time or near real-time). Alternatively and/or additionally, the cranial access assembly may include various mechanisms (e.g., linkages, motors, drives, actuators, drivers, etc.) for automatic insertion of an identified trajectory adjustment guide at the determined angular position without (or with minimal) user involvement to achieve a desired trajectory adjustment. Specifically, the cranial access assembly may include additional components having features that help automate the use of the cranial access assembly, such as actuators for mechanical movement and adjustment of the cranial access assembly. The actuators may comprise position sensors to record positional orientations of the cranial access assembly. For example, the cranial access assembly of the present disclosure may further include encoders, angular rotational recording mems devices (e.g., orientation sensor), or any other suitable device for sensing, communicating, and recording data relating to the position, angle, and rotation of various components of the cranial access assembly. In some embodiments, the position, angle, and rotation data may be mapped to a specific image set to correspond to the orientation of the cranial access assembly when the image set was taken. The position, angle, and rotation data may be used to generate a 3D map from a series of image sets, to calculate volume rendering, to determine orientation parameters for a particular angle of entry, and the like. In certain embodiments, the sensing, communicating, and recording may be activated and deactivated by a remote switch, such that a user may choose when to begin and when to terminate the collection of location data. The remote switch enables a user to capture data in an efficient manner. The positional data may, for example, be later conveyed to the actuators such that a cranial access assembly may quickly acquire the positional/angular orientation needed to find a target site (using and/or changing a trajectory adjustment guide), with subsequent use of the medical device inserted through the cranial access assembly to confirm accurate targeting.

In some embodiments, the cranial access assembly may include and/or may interface with a depth stop.

### Depth Stop

In various embodiments, a depth stop that is configured to set the depth of a medical device (e.g., a probe) is insertable within a cranial cavity may be provided. The depth stop may be removable secured to the medical device and/or one or more components of the cranial access assembly (e.g., the cranial bolt and/or the trajectory adjustment guide). FIGS. 5A-5E illustrate a floating depth stop.

As shown in FIGS. 5A-5C, depth stop 500 including a floating depth stop (FDS) lock 501 (including symmetrical locking blocks 501(a) and 501(b)) is illustrated for setting and/or adjusting the depth of a probe when inserted into a cranial cavity using the cranial access assembly discussed above. FIG. 5C illustrates an exploded view of the depth stop 500 while FIGS. 5A and 5B illustrate cross section views of the depth stop 500 from different perspectives.

As shown in FIGS. 5A and 5B, the depth stop 500 comprises a molded body that includes two rigid, opposing symmetrical and floating locking blocks 501(a) and 501(b) (collectively, FDS lock 501) disposed to form a channel 510 therebetween when positioned against each other, the channel being configured to receive a medical device. Specifically, the floating locking blocks 501(a) and 501(b) each include a semi-circular (or other shape cut-out) that can form the channel when the floating locking blocks 501(a) and 501(b) are positioned against each other. A clamp 512 (e.g., a screw) may be provided that is configured to position or push the locking blocks 501(a) and 501(b) against one another and a medical device inserted within the channel 510, and thus causing the locking blocks 501(a) and 501(b) to clamp onto and apply locking friction against the medical device. The locking blocks 501(a) and 501(b) may be formed form a rigid material that does not cause damage to the medical device when pushed against a medical device for applying the locking friction. Examples may include, without limitation, plastic, rubber, silicone, or the like. Optionally, the inner surface of the locking blocks 501(a) and 501(b) may include a sleeve (e.g., a rubber sleeve) that surrounds the channel 510. The depth stop 500 may also include other components such as a spring loaded ring button assembly 513 configured for interfacing with or locking to a cranial bolt or trajectory adjustment guide or other medical device interfacing. Push button ring button assembly 513 can lock the depth stop onto a notch in the trajectory adjustment guide or cranial bolt as shown in FIGS. 5D and 5E thereby restraining any relative linear motion of depth stop with respect to the cranial bolt or the trajectory adjustment guide.

In various embodiments, a diameter of the channel 510 (when the locking blocks are in contact with each other) is configured to be a slightly smaller (e.g., about 0.05-0.1 mm, 0.1-0.2 mm, 0.25-1 mm, about 0.5-0.75 mm smaller) than or almost similar to the outer diameter of the medical device. As such, when a medical device is inserted within the channel 510 such that the two symmetrical locking blocks are positioned against each other and around the medical device, and the clamp is tightened, the two locking blocks 501(a) and 501(b) are forced close to each other clamping the medical device preventing it from moving. The interference between the locking blocks 501(a) and 501(b) and medical device shaft 100 (e.g., as shown in FIG. 5D) is tightly controlled by controlling the size of the locking blocks with respect to the medical device shaft diameter which ensures a very consistent lock. Also, the clamp cannot be “over tightened” and damage the probe shaft because it is protected by the rigid locking blocks pressing together. The clamp may be loosened for allowing movement of the medical device to a suitable depth.

In some embodiments, the adjustable depth stop can be used with a medical device inserted into a cranial cavity using a cranial access assembly described above. For example, FIG. 5D illustrates the depth stop 500 interfacing with a trajectory adjustment guide 102 described above. As shown in FIG. 5D, medical device 100 is inserted within a cranial cavity via a trajectory adjustment guide 102 inserted within a cranial bolt 101. The trajectory adjustment guide 102 may include tabs, slots, or other suitable interfacing features 319 for interfacing with the depth stop 500. For example, as shown in FIG. 5D, the tabs 319 are configured to receive a spring loaded mating ring 513 of the depth stop 500 when the locking blocks of the depth stop 500 are positioned around the medical device 100 in order to hold the depth stop 500 at a suitable position. Other interfacing features such as without limitation, slots, clamps, pins, pinions, or the like are within the scope of this disclosure. As discussed above, the clamp 512 may be used to control a depth of the medical device 100.

FIG. 5E illustrates the depth stop 500 interfacing with a cranial bolt 101 described above. As shown in FIG. 5E, medical device 100 is inserted within the channel 110 of a cranial bolt 101. In example embodiments, the ledge 105 between the distal portion 102 and the proximal portion 104 may interface with a mating ring 513 of the depth stop 500 when the locking blocks of the depth stop 500 are positioned around the medical device 100 in order to hold the depth stop 500 at a suitable position. Other interfacing features such as without limitation, slots, clamps, pins, pinions, or the like are within the scope of this disclosure. As discussed above, the clamp 512 may be used to control a depth of the medical device 100.

As shown in FIGS. 5D and 5E, measured gradations 103 (e.g., centimeters, millimeters, inches, etc.) printed along the shaft of the medical device 100. An operator may align measured gradations on the outside of a medical device shaft with the depth stop 500, for example, by sliding a position of the depth stop along the shaft of the medical device until the desired depth measurement aligns with the distal end of the depth stop. The exposed distal portion of the medical device may then be inserted within the trajectory adjustment guide (as shown in FIG. 5D) or the cranial bolt (as shown in FIG. 5E), and the depth stop may be locked onto the trajectory adjustment guide or the cranial bolt, as discussed above.

In another example, Optionally, the medical device may be partially inserted (i.e., at less than a desired depth) within the trajectory adjustment guide or the cranial bolt before locking of the depth stop around the medical device. The medical device may then be advanced into the cranial cavity to a desired depth until the desired depth measurement aligns with the proximal end of the depth stop. The operator may align the desired depth measurement with a proximal end of the depth stop (e.g., for clearer visibility). In this example, the gradations may be applied to the shaft of the medical device to compensate for a length of the depth stop. In a further embodiment (not illustrated), the depth stop may include a window (e.g., cut-out portion, clear portion, or clear, magnified portion) for aligning a desired depth measurement while slidably positioning the depth stop. In this example, the gradations may be applied to the shaft of the medical device to compensate for the portion of the length of the depth stop from the medical device end to the depth selection window. Instead or in addition to the printed gradations, in other embodiments, the shaft of the medical device may include a series of mating points for mating with the depth stop. For example, the shaft of the medical device may include a series of bumps (e.g., every 5 millimeters, 10 millimeters, etc.) and the depth stop may include one or more mating depressions for engaging with at least one of the series of bumps. The mating points, for example, may be used to more precisely align the depth stop with a particular depth setting before locking the depth stop lock in place.

Using the depth stop lock with the guidance of the printed gradations upon the shaft of the medical device, an operator may modify the medical device length in situ. For example, during a subject operation, after applying a procedure at a first selected depth, an operator may vary the length or depth relatively rapidly to a second selected depth by unscrewing the thumb screw, moving the shaft of the medical device to a new location, and retightening the thumb screw.

It must be appreciated that the above described cranial access assembly (including the cranial bolt and the trajectory adjustment guides) has a configuration that provision for seamless integration of surgical instruments, which can be used for general surgical purposes or for a specific purpose. Therefore, the present disclosure provides for an apparatus and corresponding methods that provide a stable platform for the introduction of surgical, therapeutic or diagnostic intervention into the central nervous system (CNS), and in particular into the brain of a mammalian subject. The apparatus provides for the placement and fixation of instruments for use in neurological procedures, and in particular for neurological procedures that are performed in conjunction with preoperative or perioperative monitoring such as magnetic resonance imaging (MRI) or computed tomography (CT) imaging. Moreover, the apparatus of the present disclosure is made from a material that is compatible with MRI or CT imaging systems.

According to an embodiment, the cranial access assembly 10 has a slim profile, thereby allowing multiple bolts to be inserted into the skull at multiple trajectories that are within close proximity of one another. The skull mounted bolt is robust, accurate, and provides a seamless way to provide placement and fixation for the operation of instruments or devices. Additionally, the bolt is MRI compatible, does not create substantial artifacts during imaging, and has a slim fit to allow multiple trajectories.

According to one embodiment, the dimensions of the cranial access assembly 10 allow the device to be placed in close proximity to one or more other similar devices. This feature provides the advantageous ability of using multiple apparatus in contiguous/non-contiguous regions in a single treatment session. Accordingly, the ability of the medical provider to apply therapeutic intervention to a larger area of the CNS (e.g., the brain) in a single session is maximized. For instance, a single therapeutic session may include 2-10 skull mounted bolts (preferably 2-5 bolts) and corresponding trajectory adjustment guides that are disposed within close proximity of one another. While employing multiple bolts and corresponding trajectory adjustment guides, the inter-bolt separation is required in order to accommodate, for instance, a probe driver or a probe adapter that may be positioned over the connector portion of the bolt. Furthermore, by providing a sufficient spacing between the bolts, also provisions the surgeon with easy access to the individual bolts, as well as regions of the skull around the bolt.

According to an embodiment, components of the above discussed skull mounted cranial access devices are MRI compatible. Two or more bolts can be used in a single therapeutic session that takes place within an imaging apparatus, and two or more trajectory adjustment guides provide a wide range of trajectory adjustment options during a procedure. Accordingly, a further aspect of the present disclosure provides for methods of treating living subjects, such as human or other mammalian subjects, using a magnetic field in a magnetic resonance volume.

By one embodiment, the magnet can be positioned relative to the subject so that the magnetic resonance volume, at least partially encompasses a region of the subject to be treated. A movable applicator adapted to apply energy within an energy application zone is positioned relative to the subject so that the energy application zone intersects the magnetic resonance volume within the region of the subject requiring treatment. While the static field magnet is applying the static magnetic field in the magnetic resonance volume, radio frequency signals are applied so as to elicit magnetic resonance signals from tissues of the subject in the magnetic resonance volume. The method also includes receiving these magnetic resonance signals and deriving magnetic resonance information relative to the subject's tissues in the magnetic resonance volume from the magnetic resonance signals.

According to one embodiment, the skull mounted bolts or cranial access assemblies described above can be used in conjunction with a robotic probe driver. The robotic probe driver can align and position a tip of the probe at a certain distance from a target area (e.g., target tissues in the brain) that is to be treated, via the cranial access assembly. The probe can be used to treat various brain diseases by using thermal ablation. The diseases can range from tumors to epilepsy. According to an embodiment, the probe is aligned to the target tissue and inserted into the brain along a pre-planned trajectory until the tip reaches the target tissue. The pre-planned trajectory may be adjusted using a trajectory adjustment guide, and or the insertion of the probe may be aligned with the pre-planned trajectory using a trajectory adjustment guide intraoperatively without removal or insertion of additional cranial bolts, as discussed above. Thereafter, laser energy is transmitted through the probe and emitted from the tip inside the target area. The energy heats the tissues causing cell death. It must be appreciated that the temperature of the probe tip can be controlled using a cooling gas and thermal monitoring.

According to an embodiment, the skull mounted bolts or cranial access assemblies described above can be used in conjunction with a brain biopsy tool. The brain biopsy tool may be positioned and deployed within the target brain area via the cranial access device.

According to an embodiment, the skull mounted bolts or cranial access assemblies described above can be used in conjunction with tools for placement of electrodes (e.g., Stereoelectroencephalography (SEEG)) within the brain. An electrode can be introduced and placed within a target area (e.g., target tissues in the brain), via the cranial access device.

By one embodiment, various agents may be introduced into the CNS of the subject, and in particular the brain of a subject, using the apparatus and methods of the present disclosure. A variety of agents and compositions comprising such agents can be delivered using the device, including but not limited to chemotherapeutic agents, agents for treatment of neurodegenerative disease (e.g., neurotrophic factors or neuroprotective agents), antiepileptic agents, antidepressant agents, antipsychotic agents, anti-inflammatory agents, antifibrotic agents, antianxiolytics and the like.

By one embodiment, the agents delivered using the methods and devices of the present disclosure include gene therapy by delivery of transgenes encoding certain factors into the brain, which offers great promise for treating neurodegenerative conditions such as Alzheimer's disease, Parkinson's disease and Huntington's disease. Similarly, cell-based therapies typically require quite precise placement of the cell population into the targeted region of the CNS. Delivery of these agents requires that the therapeutic composition dosage be consistently provided at precise locations in the brain to ensure that a predictable amount of the intended cell or encoded factor be delivered only to targeted regions of the brain. Such precise delivery requires delivery vectors and cells encoding transgenes to be grafted at pre-determined sites in the target brain region. The system and methods of the present disclosure allow a precise and localized introduction of such agents into targeted regions of the brain while minimizing the invasiveness of the surgical procedure. Therefore, improvements in therapeutic efficacy can be obtained by enhancing the accurate placement of transgene-containing donor cell grafts and/or viral vectors into the brain using the apparatus and methods described herein.

Furthermore, agent delivery can be provided as a single dosage form, as a bolus or encapsulated dosage form which will release drug over time, and/or the implantation of delivery device (e.g., an osmotic pump or a catheter). Such devices for delivery of therapeutic agents that can be used in conjunction with the cranial access assemblies described herein.

In some embodiments, the devices of the present invention may operate in conjunction with a computer platform system, such as a local or remote executable software platform, or as a hosted internet or network program or portal. In certain embodiments, portions of the system may be computer operated, or in other embodiments, the entire system may be computer operated. As contemplated herein, any computing device as would be understood by those skilled in the art may be used with the system, including desktop or mobile devices, laptops, desktops, tablets, smartphones or other wireless digital/cellular phones, televisions or other thin client devices as would be understood by those skilled in the art.

The computer platform is fully capable of sending and interpreting device emissions signals as described herein throughout. For example, the computer platform can be configured to control emissions parameters such as frequency, intensity, amplitude, period, wavelength, pulsing, and the like, depending on the emissions type. The computer platform can also be configured to control the actuation of the device, such as selection and/or insertion into the cranial bolt of a trajectory adjustment guide, angulation, and locking. The computer platform can be configured to record received emissions signals, and subsequently interpret the emissions. For example, the computer platform may be configured to interpret the emissions as images and subsequently transmit the images to a digital display. The computer platform may further perform automated calculations based on the received emissions to output data such as density, distance, temperature, composition, imaging, and the like, depending on the type of emissions received. The computer platform may further provide a means to communicate the received emissions and data outputs, such as by projecting one or more static and moving images on a screen, emitting one or more auditory signals, presenting one or more digital readouts, providing one or more light indicators, providing one or more tactile responses (such as vibrations), and the like. In some embodiments, the computer platform communicates received emissions signals and data outputs in real time, such that an operator may adjust the use of the device in response to the real time communication. For example, in response to a stronger received emission, the computer platform may output a more intense light indicator, a louder auditory signal, or a more vigorous tactile response to an operator, such that the operator may adjust the device to receive a stronger signal or the operator may partially lock the device in a position that registers the strongest signal. In a further example, the computer platform may display image overlays to represent an inserted medical device in relation to a displayed ultrasound image or volume rendering (3D reconstruction) on screen.

In some embodiments, the computer platform is integrated into the devices of the present invention. For example, in some embodiments, at least one component of the computer platform described elsewhere herein is incorporated into a cranial access assembly of the present invention, such as emissions parameter controlling means, emissions recording and interpretation means, communication means for the received emissions and data outputs, and one or more features for displaying the received emissions, data, and images. Cranial access assemblies having at least one integrated computer platform component may be operable as a self-contained unit, such that additional computer platform components apart from the device itself are not necessary. Self-contained units provide a convenient means of using the devices of the present invention by performing a plurality of functions related to the devices. Self-contained units may be swappable and disposable, improving portability and decreasing the risk of contamination.

The computer operable component(s) may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

The software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a manager, expert, researcher, or other professional of the particular results. Further embodiments of such mechanisms are described elsewhere herein or may standard systems understood by those skilled in the art. As stated previously, the skull mounted bolt is robust, accurate, and provides a seamless way to provide stereotactic guidance, placement and fixation for the operation of instruments or devices. Additionally, the skull mounted bolt described herein has a slim profile, which provisions multiple bolts to be inserted into the skull within close proximity of one another.

Surgical Kits The disclosure also includes a kit comprising components useful within the methods of the disclosure and instructional material that describes, for instance, the method of using the cranial access assembly (including one or more cranial bolts and one or more trajectory adjustment guides) as described elsewhere herein. The kit may comprise components and materials useful for performing the methods of the disclosure. For instance, the kit may comprise a one or more cranial bolt(s), a fixation device, a depth stop, a reducing tube(s), a fiducial marker, probe(s) and/or one or more trajectory adjustment guides. In other embodiments, the kit may further comprise software and electronic equipment. The software and electronic equipment may be presented in a compact form for portable use.

In certain embodiments, the kit comprises instructional material. Instructional material may include a publication, a recording, a diagram, or any other medium of expression which can be used to communicate the usefulness of the device described herein. The instructional material of the kit of the disclosure may, for example, be affixed to a package which contains one or more instruments which may be necessary for the desired procedure. Alternatively, the instructional material may be shipped separately from the package, or may be accessible electronically via a communications network, such as the Internet.

In one embodiment, the disclosure includes a kit for portable use. To facilitate portable use, a kit of the present disclosure may further include a razor or clipper for removing hair from a subject, a ruler or tape measure for measuring the location of a site for incision, a surgical marker or other implement for marking the site of incision, skin preparation material (i.e., antiseptic, alcohol pads) to clean the site of incision, a scalpel to perform the incision, a drilling instrument to perforate any bone, and any additional surgical and medical elements that may be useful for such an operation, such as surgical tape, gauze, bandages, surgical thread and needle, and the like.

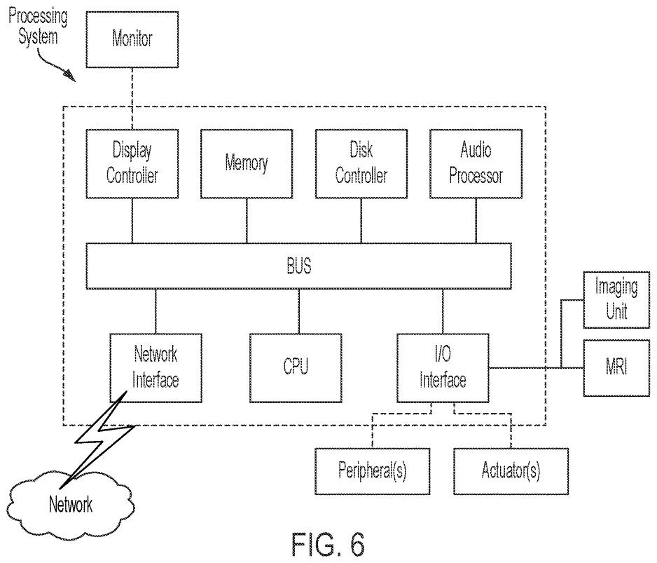

FIG. 6 illustrates an exemplary processing system, and illustrates exemplary hardware found in a controller or computing system (such as a personal computer, i.e., a laptop or desktop computer, which can embody a workstation according to this disclosure) for implementing and/or executing the processes, algorithms and/or methods described in this disclosure. A processing system in accordance with this disclosure can be implemented in one or more the components shown in FIG. 6. One or more processing systems can be provided to collectively and/or cooperatively implement the processes and algorithms discussed herein.

As shown in FIG. 6, a processing system in accordance with this disclosure can be implemented using a microprocessor or its equivalent, such as a central processing unit (CPU) and/or at least one application specific processor ASP (not shown). The microprocessor is a circuit that utilizes a computer readable storage medium, such as a memory circuit (e.g., ROM, EPROM, EEPROM, flash memory, static memory, DRAM, SDRAM, and their equivalents), configured to control the microprocessor to perform and/or control the processes and systems of this disclosure. Other storage mediums can be controlled via a controller, such as a disk controller, which can controls a hard disk drive or optical disk drive.

The microprocessor or aspects thereof, in an alternate implementations, can include or exclusively include a logic device for augmenting or fully implementing this disclosure. Such a logic device includes, but is not limited to, an application-specific integrated circuit (ASIC), a field programmable gate array (FPGA), a generic-array of logic (GAL), and their equivalents. The microprocessor can be a separate device or a single processing mechanism. Further, this disclosure can benefit from parallel processing capabilities of a multi-cored CPU.

In another aspect, results of processing in accordance with this disclosure can be displayed via a display controller to a monitor. The display controller preferably includes at least one graphic processing unit, which can be provided by a plurality of graphics processing cores, for improved computational efficiency. Additionally, an I/O (input/output) interface is provided for inputting signals and/or data from microphones, speakers, cameras, a mouse, a keyboard, a touch-based display or pad interface, etc., which can be connected to the I/O interface as a peripheral. For example, a keyboard or a pointing device for controlling parameters of the various processes and algorithms of this disclosure can be connected to the I/O interface to provide additional functionality and configuration options, or control display characteristics. Moreover, the monitor can be provided with a touch-sensitive interface for providing a command/instruction interface.

The above-noted components can be coupled to a network, such as the Internet or a local intranet, via a network interface for the transmission or reception of data, including controllable parameters. A central BUS is provided to connect the above hardware components together and provides at least one path for digital communication there between.

It will be understood that terms such as “same,” “equal,” “planar,” or “coplanar,” as used herein when referring to orientation, layout, location, shapes, sizes, amounts, or other measures do not necessarily mean an exactly identical orientation, layout, location, shape, size, amount, or other measure, but are intended to encompass nearly identical orientation, layout, location, shapes, sizes, amounts, or other measures within acceptable variations that may occur, for example, due to manufacturing processes. The term “substantially” may be used herein to emphasize this meaning, unless the context or other statements clearly indicate otherwise. For example, items described as “substantially the same,” “substantially equal,” or “substantially planar,” may be exactly the same, equal, or planar, or may be the same, equal, or planar within acceptable variations that may occur, for example, due to manufacturing processes and/or tolerances. The term “substantially” may be used to encompass this meaning, especially when such variations do not materially alter functionality.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

Some examples of embodiments of the present disclosure may be described in view of the following clauses:
Clause 1. A cranial access assembly comprising:
a cranial bolt comprising a channel configured for receiving a neurosurgical tool; and
a trajectory adjustment guide comprising:
a distal portion configured to be received within the channel at one of a plurality of angular positions, and
a proximal portion having a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel.
Clause 2. The cranial access assembly of clause 1, wherein the cranial bolt comprises a cranial bolt distal portion configured for attachment to a skull of a subject.
Clause 3. The cranial access assembly of clause 2, wherein the cranial bolt distal portion comprises threads.
Clause 4. The cranial access assembly of any of the above clauses, wherein an inner surface of the channel comprises one or more locking features that lock the distal portion at that one of the plurality of angular positions when the distal portion is inserted within the channel.
Clause 5. The cranial access assembly of clause 4, wherein the inner surface of the channel comprises a hex shape cross section that provides six angular positions of the distal portion within the channel.
Clause 6. The cranial access assembly of any of the above clauses, wherein an outer surface of the distal portion comprises one or more locking features that lock the distal portion at that one of the plurality of angular positions when inserted within the channel.
Clause 7. The cranial access assembly of any of the above clauses, wherein the trajectory adjustment guide further comprises a locking element configured to lock the distal portion at that one of the plurality of angular positions when the distal portion is inserted within the channel.
Clause 8. The cranial access assembly of clause 7, wherein the locking element comprises a screw.
Clause 9. The cranial access assembly of any of the above clauses, wherein a length of the cranial bolt is about 10 mm to about 45 mm.
Clause 10. The cranial access assembly of any of the above clauses, wherein an angular offset between the trajectory adjustment channel and the channel when the trajectory adjustment guide is at least partially inserted within the channel is about 0.5° to about 30°.
Clause 11. The cranial access assembly of any of the above clauses, wherein a diameter of the trajectory adjustment channel is configured to hold the neurosurgical tool via a friction fit.
Clause 12. The cranial access assembly of any of the above clauses, wherein at least one of the cranial bolt or the trajectory adjustment guide comprise one or more interfacing features configured to interface with a depth stop that is configured to set a depth of the neurological tool within a cranial cavity of a subject.
Clause 13. A trajectory adjustment guide for adjusting a trajectory of a neurosurgical tool, the trajectory adjustment guide comprising:
a distal portion configured to be received within a channel of a cranial bolt at one of a plurality of angular positions, the cranial bolt being affixed to a skull of a subject along a pre-planned trajectory; and
a proximal portion having a longitudinal axis that is angularly offset with respect to a longitudinal axis of the distal portion such that the distal portion and the proximal portion define a trajectory adjustment channel configured for receiving a neurosurgical tool along a trajectory that is angularly offset with respect to the channel when the trajectory adjustment guide is at least partially inserted within the channel.
Clause 14. The trajectory adjustment guide of clause 13, wherein an outer surface of the distal portion comprises one or more locking features that lock the distal portion at that one of the plurality of angular positions when inserted within the channel of the cranial bolt.
Clause 15. The trajectory adjustment guide of any of the trajectory adjustment guide clauses, wherein the trajectory adjustment guide further comprises a locking element configured to lock the distal portion at that one of the plurality of angular positions when the distal portion is inserted within the channel of the cranial bolt.
Clause 16. The trajectory adjustment guide of clause 15, wherein the locking element comprises a screw.
Clause 17. The trajectory adjustment guide of any of the above trajectory adjustment guide clauses, wherein an angular offset between the trajectory adjustment channel and the channel when the trajectory adjustment guide is at least partially inserted within the channel is about 0.5° to about 30°.
Clause 18. The trajectory adjustment guide of any of the above trajectory adjustment guide clauses, wherein a diameter of the trajectory adjustment channel is configured to hold the neurosurgical tool via a friction fit.
Clause 19. The trajectory adjustment guide of any of the above trajectory adjustment guide clauses, wherein the trajectory adjustment guide further comprises one or more interfacing features configured to interface with a depth stop that is configured to set a depth of the neurological tool within a cranial cavity of a subject.
Clause 20. The trajectory adjustment guide of clause 19, wherein the one or more interfacing features comprise a slot configured to receive an interface ring of the depth stop.
Clause 21. A floating depth stop for controlling a depth of a neurosurgical tool inserted within a cranial cavity via the cranial access assembly of any of the above clauses, the depth stop comprising two symmetrical locking blocks that define a depth stop channel.
Clause 22. The floating depth stop of clause 21, further comprising an interfacing spring loaded ring configured to interface with at least one of the cranial bolt or the trajectory adjustment guide.
Clause 23. The floating depth stop of any of the depth stop clauses, wherein each of the two symmetrical locking blocks comprises a sleeve surrounding the depth stop channel.
Clause 24. The floating depth stop of any of the depth stop clauses, further comprising a clamp configured for pushing the deformable locking elements around the neurosurgical tool within the depth stop channel to lock an insertion depth of the neurosurgical tool.
Clause 25. The floating depth stop of any of the depth stop clauses, wherein the symmetrical locking blocks are formed from a rigid material to prevent overtightening of the depth stop channel around the neurosurgical tool.
Clause 26. A surgical kit comprising the cranial access assembly of any of the above clauses; and
a plurality of trajectory adjustment guides, each having a different angular offset between the channel and the corresponding trajectory adjustment channel.
Clause 27. A surgical kit of clause 26, further comprising a floating depth stop configured to set a depth of insertion of the neurosurgical tool.
Clause 28. The surgical kit of clause 27, further comprising the neurosurgical tool.
Clause 29. The surgical kit of any of the above surgical kit clauses, further comprising a plurality of reducing tubes configured to reduce a diameter of the trajectory adjustment channel.
Clause 30. The surgical kit of any of the above surgical kit clauses, further comprising a plurality of cranial bolts each having a different channel diameter capable of accepting trajectory adjustment guides of varying diameters.
Clause 31. A method for adjusting the trajectory of a neurosurgical tool using a cranial access assembly, the method comprising:
affixing a cranial bolt to a skull of a subject along a first trajectory, the cranial bolt comprising a channel configured for receiving a neurosurgical tool;
inserting the neurosurgical tool within the channel the cranial bolt;
determining a trajectory of the neurosurgical tool;
in response to determining that the trajectory does not align with the first trajectory or will not cause the neurosurgical tool to reach a target site within a cranial cavity, identifying from a plurality of trajectory adjustment guides, a trajectory adjustment guide for adjusting the trajectory to align with the first trajectory or cause the neurosurgical tool to reach the target site within the cranial cavity, the identified trajectory guide comprising:
a distal portion configured to be received within the channel at one of a plurality of angular positions that is configured to adjust trajectory, and
a proximal portion having a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel;
inserting the identified trajectory adjustment guide within the channel; and
advancing the neurosurgical tool within the trajectory adjustment channel.
Clause 32. The method of clause 31, further comprising controlling a depth of the neurosurgical tool using a floating depth stop.

1. A cranial access assembly comprising:
a cranial bolt comprising a channel configured for receiving a neurosurgical tool; and
a trajectory adjustment guide comprising:
a distal portion configured to be received within the channel at one of a plurality of angular positions, and
a proximal portion having a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel.

2. The cranial access assembly of claim 1, wherein the cranial bolt comprises a cranial bolt distal portion configured for attachment to a skull of a subject.

3. The cranial access assembly of claim 2, wherein the cranial bolt distal portion comprises threads.

4. The cranial access assembly of claim 1, wherein an inner surface of the channel comprises one or more locking features that lock the distal portion at that one of the plurality of angular positions when the distal portion is inserted within the channel.

5. The cranial access assembly of claim 4, wherein the inner surface of the channel comprises a hex shape cross section that provides six angular positions of the distal portion within the channel.

6. The cranial access assembly of claim 1, wherein an outer surface of the distal portion comprises one or more locking features that lock the distal portion at that one of the plurality of angular positions when inserted within the channel.

7. The cranial access assembly of claim 1, wherein the trajectory adjustment guide further comprises a locking element configured to lock the distal portion at that one of the plurality of angular positions when the distal portion is inserted within the channel.

8. The cranial access assembly of claim 7, wherein the locking element comprises a screw.

9. The cranial access assembly of claim 1, wherein a length of the cranial bolt is about 10 mm to about 45 mm.

10. The cranial access assembly of claim 1, wherein an angular offset between the trajectory adjustment channel and the channel when the trajectory adjustment guide is at least partially inserted within the channel is about 0.5° to about 30°.

11. The cranial access assembly of claim 1, wherein a diameter of the trajectory adjustment channel is configured to hold the neurosurgical tool via a friction fit.

12. The cranial access assembly of claim 1, wherein at least one of the cranial bolt or the trajectory adjustment guide comprise one or more interfacing features configured to interface with a depth stop that is configured to set a depth of the neurological tool within a cranial cavity of a subject.

13. A trajectory adjustment guide for adjusting a trajectory of a neurosurgical tool, the trajectory adjustment guide comprising:
a distal portion configured to be received within a channel of a cranial bolt at one of a plurality of angular positions, the cranial bolt being affixed to a skull of a subject along a pre-planned trajectory; and
a proximal portion having a longitudinal axis that is angularly offset with respect to a longitudinal axis of the distal portion such that the distal portion and the proximal portion define a trajectory adjustment channel configured for receiving a neurosurgical tool along a trajectory that is angularly offset with respect to the channel when the trajectory adjustment guide is at least partially inserted within the channel.

14. The trajectory adjustment guide of claim 13, wherein an outer surface of the distal portion comprises one or more locking features that lock the distal portion at that one of the plurality of angular positions when inserted within the channel of the cranial bolt.

15. The trajectory adjustment guide of claim 13, wherein the trajectory adjustment guide further comprises a locking element configured to lock the distal portion at that one of the plurality of angular positions when the distal portion is inserted within the channel of the cranial bolt.

16. The trajectory adjustment guide of claim 15, wherein the locking element comprises a screw.

17. The trajectory adjustment guide of claim 13, wherein an angular offset between the trajectory adjustment channel and the channel when the trajectory adjustment guide is at least partially inserted within the channel is about 0.5° to about 30°.

18. The trajectory adjustment guide of claim 13, wherein a diameter of the trajectory adjustment channel is configured to hold the neurosurgical tool via a friction fit.

19. The trajectory adjustment guide of claim 13, wherein the trajectory adjustment guide further comprises one or more interfacing features configured to interface with a depth stop that is configured to set a depth of the neurological tool within a cranial cavity of a subject.

20. The trajectory adjustment guide of claim 19, wherein the one or more interfacing features comprise a slot configured to receive an interface ring of the depth stop.

21. A floating depth stop for controlling a depth of a neurosurgical tool inserted within a cranial cavity via the cranial access assembly of claim 1, the depth stop comprising two symmetrical locking blocks that define a depth stop channel.

22.-30. (canceled)

31. A method for adjusting the trajectory of a neurosurgical tool using a cranial access assembly, the method comprising:
affixing a cranial bolt to a skull of a subject along a first trajectory, the cranial bolt comprising a channel configured for receiving a neurosurgical tool;
inserting the neurosurgical tool within the channel the cranial bolt;
determining a trajectory of the neurosurgical tool;
in response to determining that the trajectory does not align with the first trajectory or will not cause the neurosurgical tool to reach a target site within a cranial cavity, identifying from a plurality of trajectory adjustment guides, a trajectory adjustment guide for adjusting the trajectory to align with the first trajectory or cause the neurosurgical tool to reach the target site within the cranial cavity, the identified trajectory guide comprising:
a distal portion configured to be received within the channel at one of a plurality of angular positions that is configured to adjust trajectory, and
a proximal portion having a longitudinal axis that is angularly offset with respect to a longitudinal axis of the distal portion such that the distal portion and the proximal portion define a trajectory adjustment channel that is angularly offset with respect to the channel when the trajectory adjustment guide is at least partially inserted within the channel;
inserting the identified trajectory adjustment guide within the channel; and
advancing the neurosurgical tool within the trajectory adjustment channel.

32. (canceled)
